(19) United States
(12) Patent Application Publication
(10) Pub. No.: US 2025/0312286 A1
(43) Pub. Date: Oct. 9, 2025

(54) METHOD FOR PREPARING MICROCAPSULE, MICROCAPSULE AND COMPOSITION CONTAINING THE SAME

(71) Applicants: XIAMEN KINGDOMWAY GROUP COMPANY, Xiamen (CN); XIAMEN KINGDOMWAY BIOTECH. CO., LTD., Xiamen (CN)

(72) Inventors: Fangfang CHEN, Xiamen (CN); Huaying LIU, Xiamen (CN); Dan LI, Xiamen (CN); Murong LIN, Xiamen (CN); Jinhong LIU, Xiamen (CN); Wenji WANG, Xiamen (CN)

(21) Appl. No.: 18/814,339

(22) Filed: Aug. 23, 2024

(30) Foreign Application Priority Data
Apr. 7, 2024 (CN) 202410411027.3

(51) Int. Cl. A61K 9/50; A61K 45/06

(52) U.S. Cl. CPC A61K 9/5089; A61K 9/5015; A61K 9/5052; A61K 9/5073; A61K 45/06

(57) ABSTRACT

The present invention belongs to the field of food or medicine, and relates to a method for preparing microcapsule, a microcapsule and a composition containing the same. The present invention also relates to a wall material dispersion combination. Specifically, the present invention relates to a method for preparing microcapsule, wherein the microcapsule includes a wall material and a core material, and the wall material includes a first wall material and a second wall material; the method includes a step of preparing a first dispersion containing the first wall material and a second dispersion containing the second wall material; wherein the first dispersion is subjected to a heat treatment, the heat treatment includes a first heating stage and a second heating stage, and the temperature of the first heating stage is lower than the temperature of the second heating stage; and the second dispersion is not subjected to a heat treatment, or is subjected to a heat treatment at a temperature of not exceeding 50° C., or the second dispersion is subjected to a heat treatment at a temperature that is lower than the temperature of the heat treatment to which the first dispersion is subjected. The microcapsule prepared by the present invention has good stability and/or water solubility.

### TECHNICAL FIELD

The present invention belongs to the field of food or medicine, and relates to a method for preparing microcapsule, a microcapsule and a composition containing the same. The present invention also relates to a wall material dispersion combination.

### BACKGROUND ART

Nutrients are substances that need to be taken in from the external environment in order to maintain all life activities and processes such as reproduction, growth, development and survival of the body, including fat-soluble nutrients and water-soluble nutrients.

Fat-soluble nutrients refer to nutrients that have hydrophobic properties and are more soluble in organic solvents or cell membranes than in aqueous solutions, mainly including fat-soluble vitamins, fat-soluble quasi-vitamins, carotenoids, polyunsaturated fatty acids, and monounsaturated fatty acids, etc. Fat-soluble nutrients play an important role in the growth, metabolism, and development of the body.

Water-soluble nutrients refer to nutrients that can be dissolved in water, including water-soluble vitamins, mineral ions, nattokinase, pyrroloquinoline quinone sodium salt, active folic acid (calcium L-5-methyltetrahydrofolate), phosphatidylserine, glutathione, nicotinamide adenine mononucleotide, nicotinamide adenine dinucleotide, etc.

Most nutrients are basically very unstable substances and are easily affected by the external environments (temperature, light, oxygen, etc.), processing and storage conditions, and the digestive tract environments (pH value, enzymes, other substances), and are often not suitable for adding directly to feed, food or medicine. In addition, for fat-soluble nutrients, due to their water insolubility, their absorption in the body is limited and their bioavailability is low. Therefore, many researchers have developed various methods to improve the stability and/or water solubility of nutrients. Usually, microencapsulation technology can be used to add excipients to nutrients and encapsulate them to form microcapsules.

Microcapsule refers to a micro container or package with a polymer shell, which generally has a size ranging from 5 to 200 μm and has various shapes, depending on the raw materials and preparation method. The process of preparing microcapsules is called microencapsulation. Microencapsulation technology refers to a technology that embeds a solid, liquid or gas in tiny and sealed capsules so that it can be released at a controlled rate only under specific conditions. The embedded substance is called core material, and the substance that embeds the core material to achieve microencapsulation is called wall material.

The method adopted in the prior art to improve the stability of nutrient microcapsules is usually to add an antioxidant to the core material or wall material. For example, CN115005446A relates to an organic DHA microcapsule powder and its preparation method, in which the DHA powder is composed of the following components in mass percentages: 25.0% to 30.0% of DHA, 65% to 72% of dispersant, 0.1% to 0.3% of acidity regulator, 0.1% to 0.3% of water-soluble antioxidant, 1.5% to 3.4% of oil-soluble antioxidant, and 0.5% to 1% of anti-caking agent. CN114158732A relates to a polyunsaturated fatty acid triglyceride microcapsule powder and its preparation method, in which the microcapsule contains a wall material carbohydrate, a core material polyunsaturated fatty acid oil, an emulsifier, a first oxidant and a second antioxidant, and the triglyceride content of the polyunsaturated fatty acid oil is 65% to 100%. However, because nutrients are extremely unstable, the granulation process involves strong mechanical action, the contact surface between the core material and the external environment is large, and the nutrients are easily deteriorated, the technical solution of adding antioxidants is still not ideal.

Therefore, it is of great significance for the application of nutrients to provide a preparation method of nutrient microcapsules that improves the stability of nutrients in adverse environments and ensures good water solubility of nutrient microcapsules.

### CONTENTS OF THE PRESENT INVENTION

The inventors of the present invention have obtained a microcapsule through in-depth research and creative work. The inventors of the present invention surprisingly found that the microcapsules have good stability and/or water solubility, and achieve the compatibility of stability and water solubility. Therefore, the following invention is provided:

One aspect of the present invention relates to a method of preparing a microcapsule,
the microcapsule comprises a wall material and a core material, and the wall material comprises a first wall material and a second wall material;
the method comprises a step of preparing a first dispersion containing the first wall material and a second dispersion containing the second wall material;
wherein,
the first dispersion is subjected to a heat treatment, the heat treatment comprises a first heating stage and a second heating stage, and the temperature of the first heating stage is lower than the temperature of the second heating stage; and
the second dispersion is not subjected to a heat treatment, or is subjected to a heat treatment at a temperature of not more than 50° C. (e.g., not more than 45° C., not more than 40° C., not more than 35° C.; preferably, higher than room temperature, such as 25° C.), or the second dispersion is subjected to a heat treatment at a temperature that is lower than the temperature of the heat treatment to which the first dispersion is subjected.

In some embodiments of the present invention, in the preparation method, the weight of the first wall material is less than or equal to the weight of the second wall material.

In some embodiments of the present invention, in the preparation method, the weight ratio of the first wall material to the second wall material is 1:(0.5-3), preferably 1:(1-3), 1:(1.2-3) or 1:(1.5-3).

In some embodiments of the present invention, in the preparation method, the temperature of the heat treatment to which the first dispersion is subjected is 50° C. to 120° C., 50° C. to 90° C., 50° C. to 80° C., 55° C. to 65° C.° C., 55° C. to 60° C. or 60° C. to 65° C.

In some embodiments of the present invention, in the preparation method, the heating time of the first heating stage is longer than the heating time of the second heating stage.

In some embodiments of the present invention, in the preparation method, the temperature of the first heating stage is 50° C. to 90° C., 50° C. to 80° C., 55° C. to 65° C., 55° C. to 60° C. or 60° C. to 65° C.; and/or the temperature of the second heating stage is 90° C. to 120° C., 90° C. to 110° C., 100° C. to 120° C., 105° C. to 115° C., 110° C. to 120° C., or 100° C. to 110° C.

In some embodiments of the present invention, in the preparation method,
the temperature of the first heating stage is 50° C. to 90° C.; and/or
the temperature of the second heating stage is 100° C. to 120° C., 105° C. to 115° C., 110° C. to 120° C., or 100° C. to 110° C.

In some embodiments of the present invention, in the preparation method,
the temperature of the first heating stage is 50° C. to 80° C.; and/or
the temperature of the second heating stage is 90° C. to 120° C., 90° C. to 110° C., 100° C. to 120° C., 105° C. to 115° C., 110° C. to 120° C., or 100° C. to 110° C.

In some embodiments of the present invention, in the preparation method,
the temperature of the first heating stage is 55° C. to 65° C.; and/or
the temperature of the second heating stage is 90° C. to 120° C., 90° C. to 110° C., 100° C. to 120° C., 105° C. to 115° C., 110° C. to 120° C., or 100° C. to 110° C.

In some embodiments of the present invention, in the preparation method,
the temperature of the first heating stage is 55° C. to 60° C.; and/or
the temperature of the second heating stage is 90° C. to 120° C., 90° C. to 110° C., 100° C. to 120° C., 105° C. to 115° C., 110° C. to 120° C., or 100° C. to 110° C.

In some embodiments of the present invention, in the preparation method,
the temperature of the first heating stage is 60° C. to 65° C.; and/or
the temperature of the second heating stage is 90° C. to 120° C., 90° C. to 110° C., 100° C. to 120° C., 105° C. to 115° C., 110° C. to 120° C., or 100° C. to 110° C.

In some embodiments of the present invention, in the preparation method,
the heating time of the first heating stage is 30 to 180 minutes, 30 to 120 minutes, 30 to 90 minutes, 30 to 60 minutes, 60 to 90 minutes, or 25 to 35 minutes; and/or
the heating time of the second heating stage is 5 to 25 minutes, 5 to 20 minutes, 5 to 15 minutes, 5 to 10 minutes, 10 to 25 minutes, 10 to 20 minutes, 10 to 15 minutes, or 8 to 12 minutes.

In some embodiments of the present invention, in the preparation method,
the heating time of the first heating stage is 30 to 180 minutes; and/or
the heating time of the second heating stage is 5 to 25 minutes, 5 to 20 minutes, 5 to 15 minutes, 5 to 10 minutes, 10 to 25 minutes, 10 to 20 minutes, 10 to 15 minutes, or 8 to 12 minutes.

In some embodiments of the present invention, in the preparation method,
the heating time of the first heating stage is 30 to 120 minutes; and/or
the heating time of the second heating stage is 5 to 25 minutes, 5 to 20 minutes, 5 to 15 minutes, 5 to 10 minutes, 10 to 25 minutes, 10 to 20 minutes, 10 to 15 minutes, or 8 to 12 minutes.

In some embodiments of the present invention, in the preparation method,
the heating time of the first heating stage is 30 to 90 minutes; and/or
the heating time of the second heating stage is 5 to 25 minutes, 5 to 20 minutes, 5 to 15 minutes, 5 to 10 minutes, 10 to 25 minutes, 10 to 20 minutes, 10 to 15 minutes, or 8 to 12 minutes.

In some embodiments of the present invention, in the preparation method,
the heating time of the first heating stage is 30 to 60 minutes; and/or
the heating time of the second heating stage is 5 to 25 minutes, 5 to 20 minutes, 5 to 15 minutes, 5 to 10 minutes, 10 to 25 minutes, 10 to 20 minutes, 10 to 15 minutes, or 8 to 12 minutes.

In some embodiments of the present invention, in the preparation method,
the heating time of the first heating stage is 60 to 90 minutes; and/or
the heating time of the second heating stage is 5 to 25 minutes, 5 to 20 minutes, 5 to 15 minutes, 5 to 10 minutes, 10 to 25 minutes, 10 to 20 minutes, 10 to 15 minutes, or 8 to 12 minutes.

In some embodiments of the present invention, in the preparation method,
the heating time of the first heating stage is 25 to 35 minutes; and/or
the heating time of the second heating stage is 5 to 25 minutes, 5 to 20 minutes, 5 to 15 minutes, 5 to 10 minutes, 10 to 25 minutes, 10 to 20 minutes, 10 to 15 minutes, or 8 to 12 minutes.

In some embodiments of the present invention, in the preparation method,
the temperature of the first heating stage is 55° C. to 65° C. or 55° C. to 60° C., and the temperature of the second heating stage is 105° C. to 115° C.; and
the heating time of the first heating stage is 25 to 35 minutes, and the heating time of the second heating stage is 8 to 12 minutes.

In some embodiments of the present invention, in the preparation method, the wall material is composed of a first wall material and a second wall material.

In some embodiments of the present invention, in the preparation method, the first wall material and the second wall material are the same or different.

In some embodiments of the present invention, in the preparation method, the wall material of the microcapsule accounts for 40% to 99%, preferably 50% to 85%, more preferably 65% to 75% or 60% to 70%, such as 60% to 65%, 65% to 70%, 60%, 61%, 62%, 63%, 64%, 65%, 66%, 67%, 68%, 69%, or 70% of the weight of the microcapsule.

In some embodiments of the present invention, the preparation method, the first wall material and the second wall material independently comprise a protein-based wall material and/or a carbohydrate-based wall material;
preferably, the protein-based wall material is one or more selected from the group consisting of alkali metal caseinate, whey protein, soy protein, wheat protein and corn protein; preferably, the alkali metal caseinate is one or more selected from the group consisting of sodium caseinate, potassium caseinate and calcium caseinate;
preferably, the carbohydrate-based wall material is one or more selected from the group consisting of starch, glucose, xylose, maltodextrin, maltose syrup, fructose, galactose, lactose, corn syrup, mannose and isomaltulose; preferably, the starch is a corn starch; preferably, the starch is a modified starch; preferably, the modified starch is one or more selected from the group consisting of sodium octenyl starch succinate and calcium octenyl starch succinate;
preferably, the first wall material and the second wall material also independently contain a first antioxidant; preferably, the first antioxidant is a water-soluble antioxidant, preferably one or more selected from the group consisting of tea polyphenols, ascorbic acid, erythorbic acid, sodium ascorbate and sodium erythorbate.

In some embodiments of the present invention, in the preparation method, the first wall material comprises the protein-based wall material and carbohydrate-based wall material, and the second wall material comprises the protein-based wall material and carbohydrate-based wall material.

In some embodiments of the present invention, in the preparation method, the first wall material comprises the protein-based wall material and carbohydrate-based wall material, and the second wall material comprises the protein-based wall material.

In some embodiments of the present invention, in the preparation method, the first wall material comprises the protein-based wall material and carbohydrate-based wall material, and the second wall material comprises the carbohydrate-based wall material.

In some embodiments of the present invention, in the preparation method, the first wall material is composed of the protein-based wall material and carbohydrate-based wall material, and the second wall material is composed of the protein-based wall material and carbohydrate-based wall material.

In some embodiments of the present invention, in the preparation method, the first wall material is composed of the protein-based wall material or carbohydrate-based wall material, and the second wall material is composed of the protein-based wall material.

In some embodiments of the present invention, in the preparation method, the first wall material is composed of the protein-based wall material or carbohydrate-based wall material, and the second wall material is composed of the carbohydrate-based wall material.

In some embodiments of the present invention, in the preparation method, the first wall material comprises the protein-based wall material, the carbohydrate-based wall material and the first antioxidant, and the second wall material comprises the protein-based wall material, the carbohydrate-based wall material and the first antioxidant.

In some embodiments of the present invention, in the preparation method, the first wall material comprises the protein-based wall material, the carbohydrate-based wall material and the first antioxidant, and the second wall material comprises the protein-based wall material and the first antioxidant.

In some embodiments of the present invention, in the preparation method, the first wall material comprises the protein-based wall material, the carbohydrate-based wall material and the first antioxidant, and the second wall material comprises the carbohydrate-based wall material and the first antioxidant.

In some embodiments of the present invention, in the preparation method, the first wall material consists of the protein-based wall material and the carbohydrate-based wall material and comprises the first antioxidant, and the second wall material consists of the protein-based wall material and the carbohydrate-based wall material and comprises the first antioxidant.

In some embodiments of the present invention, in the preparation method, the first wall material consists of the protein-based wall material and the carbohydrate-based wall material and comprises the first antioxidant, and the second wall material consists of the protein-based wall material and comprises the first antioxidant.

In some embodiments of the present invention, in the preparation method, the first wall material consists of the protein-based wall material and the carbohydrate-based wall material and comprises the first antioxidant, and the second wall material consists of the carbohydrate-based wall material and comprises the first antioxidant.

In some embodiments of the present invention, in the preparation method, the first dispersion and the second dispersion are independently solutions or emulsions.

In some embodiments of the present invention, in the preparation method, the core material comprises an active ingredient, such as a water-soluble active ingredient and/or a fat-soluble active ingredient;

preferably, the active ingredient is a nutrient, such as a water-soluble nutrient and/or a fat-soluble nutrient;

preferably, the core material further comprises one or more selected from the group consisting of emulsifier, second antioxidant and carrier oil.

In some embodiments of the present invention, in the preparation method, the emulsifier is at least one selected from the group consisting of monoglycerides, Spans, polyglycerol esters and phospholipids, wherein monoglycerides are preferably at least one of glyceryl monostearate, glyceryl monooleate and glyceryl monolaurate; the Spans are preferably at least one of Span-20, Span-40, Span-60, Span-80 and Span-85; the polyglycerol esters are preferably at least one of triglycerol monostearate, hexaglycerol monooleate and decaglycerol decaoleate; the phospholipids are preferably at least one of soy phospholipid, lecithin, cephalin and phosphatidylserine.

In some embodiments of the present invention, in the preparation method, the second antioxidant is an oil-soluble antioxidant, and is at least one selected from the group consisting of tocopherol, ascorbyl palmitate, rosemary extract, phospholipid, butylhydroxyanisole (BHA), dibutylhydroxytoluene (BHT) and tert-butylhydroquinone (TBHQ), and the tocopherol is at least one selected from the group consisting of α-tocopherol, β-tocopherol, γ-tocopherol, 8-tocopherol and tocotrienol, and the phospholipid is at least one selected from the group consisting of soybean phospholipid, lecithin, cephalin and phosphatidylserine.

In some embodiments of the present invention, in the preparation method, the carrier oil is one or more selected from the group consisting of soybean oil, palm kernel oil, cottonseed oil, rapeseed oil, sunflower oil, coconut oil, corn oil, sesame oil, rice bran oil, castor oil, olive oil, flax oil, safflower oil, peanut oil, and medium-chain fatty glyceride, which can be mixed in any proportion.

In some embodiments of the present invention, in the preparation method, the core material accounts for 1% to 60% of the weight of the microcapsule.

In some embodiments of the present invention, in the preparation method, the active ingredient (such as nutrients) account for 1% to 50% of the weight of the microcapsule.

In some embodiments of the present invention, in the preparation method, the microcapsule consists of the wall material and the core material.

In some embodiments of the present invention, in the preparation method, the microcapsule consists of the first wall material, the second wall material and the core material.

In some embodiments of the present invention, in the preparation method,
the fat-soluble nutrient is one or more selected from the group consisting of fat-soluble vitamin, fat-soluble quasi-vitamin, carotenoid, polyunsaturated fatty acid and monounsaturated fatty acid;
preferably, the fat-soluble vitamin is one or more selected from the group consisting of vitamin A, vitamin D, vitamin E and vitamin K;
preferably, the fat-soluble quasi-vitamin is at least one selected from the group consisting of coenzyme Q10, taurine and carnitine; wherein the coenzyme Q10 comprises one or more selected from the group consisting of oxidized coenzyme Q10 and reduced coenzyme Q10;
preferably, the carotenoid is one or more selected from the group consisting of β-carotene, β-zeaxanthin, astaxanthin, canthaxanthin, lycopene, lutein, nobiletin and zeaxanthin;
preferably, the polyunsaturated fatty acid is one or more selected from the group consisting of docosahexaenoic acid, docosapentaenoic acid, eicosapentaenoic acid, eicosatetraenonic acid, linoleic acid and linolenic acid; the polyunsaturated fatty acid can be added in the form of an oil containing the above substance, for example, such as in the form of fish oil, algae oil or vegetable oil;
preferably, the monounsaturated fatty acid is one or more selected from the group consisting of oleic acid, myristic acid, palmitoleic acid, trans-oleic acid, ricinoleic acid, erucic acid, and cetoleic acid; the monounsaturated fatty acid can be added in the form of an oil containing the above substance, such as in the form of fish oil, algae oil or vegetable oil.

In some embodiments of the present invention, in the preparation method,
the water-soluble nutrient is one or more selected from the group consisting of water-soluble vitamin, mineral ion, nattokinase, S-adenosylmethionine, glyceric acid, pyrroloquinoline quinone sodium salt, phosphatidylserine, glutathione, nicotinamide, nicotinamide adenine mononucleotide, nicotinamide ribose, and nicotinamide adenine dinucleotide;
preferably, the water-soluble vitamin is one or more selected from the group consisting of vitamin C and vitamin B family;
preferably, the vitamin B family is one or more selected from the group consisting of vitamin B1, vitamin B2 (riboflavin), vitamin B3 (niacin), vitamin B5 (pantothenic acid), vitamin B6, folic acid, vitamin B12, niacin and lipoic acid; preferably, the folic acid is an active folic acid, such as calcium L-5-methyltetrahydrofolate;
preferably, the mineral ion is one or more selected from the group consisting of sodium ion, potassium ion, calcium ion, zinc ion, magnesium ion and iron ion;
the glutathione is one or more selected from the group consisting of oxidized glutathione and reduced glutathione;
the nicotinamide adenine mononucleotide is one or more selected from the group consisting of reduced β-nicotinamide mononucleotide (NMNH) and oxidized β-nicotinamide mononucleotide (NMN);
the nicotinamide adenine dinucleotide is one or more selected from the group consisting of reduced nicotinamide adenine dinucleotide (NADH) and oxidized nicotinamide adenine dinucleotide (NAD+).

The microcapsule of the present invention may also comprise additives such as flavoring agent, colorant, glidant, etc. The specific optional varieties of the additives such as flavoring agent, colorant, glidant, etc., and their addition amounts and addition methods are all well known in this art and will not be described in detail here.

In some embodiments of the present invention, the preparation method further comprises a step of preparing the microcapsule from the first dispersion, the second dispersion and the core material; preferably, it comprises the following steps:
A. the first dispersion that has been subjected to the heat treatment is mixed with the core material, and undergoes emulsification;
B. the second dispersion that has been subjected or not been subjected to the heat treatment is added, and undergoes emulsification; and
C. granulation and drying are performed to obtain the microcapsule;
preferably, it further comprises the following step:
D. the microcapsule of step C is embedded to obtain a double-coated microcapsule.

In some embodiments of the present invention, in the preparation method,
the emulsification in step A and step B is independently performed by high-speed shearing method, high-pressure homogenization method, micro-jet method or ultrasonic method; and/or
the granulation and drying in step C are performed by spray cooling method, spray drying method or spray fluidized bed drying method to obtain the microcapsule.

In some embodiments of the present invention, in the preparation method,
the microcapsule comprises a wall material and a core material, and the wall material comprises a first wall material and a second wall material;
the method comprises the following steps:
(1) a first dispersion containing the first wall material and a second dispersion containing the second wall material are prepared;
wherein, the first dispersion is heated at 55° C. to 60° C. for 25 to 35 minutes, and then heated at 105° C. to 115° C. for 8 to 12 minutes; and the second dispersion is not heated;
(2) the first dispersion is mixed with the core material, and undergoes emulsification;
(3) the second dispersion is added, and undergoes emulsification; and
(4) granulation and drying are performed to obtain the microcapsule;
wherein,
the core material comprises 35 to 45 parts by weight of reduced coenzyme Q10 and appropriate amounts of an emulsifier and an oil-soluble antioxidant;
the first dispersion comprises 15 to 20 parts by weight of sodium octenyl starch succinate, 4 to 7 parts by weight of sodium caseinate, and appropriate amounts of a carbohydrate-based wall material (preferably corn syrup, etc.), an antioxidant (preferably sodium ascorbate, etc.) and water; and
the second dispersion comprises 20 to 25 parts by weight of sodium octenyl starch succinate and appropriate amounts of a carbohydrate-based wall material (preferably corn syrup, etc.), an antioxidant (preferably sodium ascorbate, etc.) and water.

In some embodiments of the present invention, in the preparation method, in step (A) or step (2), the heated first dispersion is cooled to room temperature and then mixed with the core material.

In some embodiments of the present invention, in the preparation method, in step (A) or step (2), the emulsification is performed for a time of 10 min to 45 min.

In some embodiments of the present invention, in the preparation method, in step (B) or step (3), the emulsification time is 5 min to 30 min.

The emulsification method is not particularly limited and can be any conventional emulsification method, such as high-speed shearing method, high-pressure homogenization method, micro-jet method or ultrasonic method, etc.

The granulation method is not particularly limited and can be any conventional granulation method, such as spray cooling method, spray drying method or spray fluidized bed drying method, etc.

In some embodiments of the present invention, in the preparation method, after step (C) or step (4), the microcapsule may be subjected to a secondary embedding to obtain a double-coated microcapsule. Steps (A) to (C) or steps (2) to (4) can be considered as first embedding.

Without being limited by any theory, the secondary embedding refers to adsorbing a wall material such as corn starch on the first-coated microcapsule to obtain a double-coated microcapsule. After the secondary embedding, the stability performance was further improved. Examples of secondary embedding can be found in Example 8 of the present invention.

Another aspect of the present invention relates to a microcapsule, which is prepared by the preparation method according to any one of the items of the present invention.

Yet another aspect of the present invention relates to a composition, which comprises the microcapsule of the present invention and one or more pharmaceutically or bromatologically acceptable auxiliary materials;
preferably, the composition is a tablet, soft capsule, hard capsule, powder or pill.

The auxiliary materials include common auxiliary materials, such as glidant (potassium ferrocyanide, sodium aluminosilicate, tricalcium phosphate, silicon dioxide, microcrystalline cellulose, aluminum silicate, calcium aluminum silicate, calcium silicate or calcium stearate), flavoring agent, colorant, plasticizer, etc.

The product form of the composition may be a food additive, a feed additive, a food, a feed, a medicine or a cosmetic, etc.

Yet another aspect of the present invention relates to a dispersion combination of microcapsule wall materials, including a first dispersion and a second dispersion, and the first dispersion and the second dispersion being mixed or not mixed;
wherein,
the first dispersion is the first dispersion according to any one of the items of the present invention; and
the second dispersion is the second dispersion according to any one of the items of the present invention.

In the present invention, unless otherwise specified, the “first” (e.g., first wall material, first dispersion, first antioxidant, etc.) or the “second” (e.g., second wall material, second dispersion, second antioxidant, etc.) are only used for reference distinction and do not have any special sequential meaning.

### Beneficial Effects of the Present Invention

The present invention achieves one or more of the following technical effects:
(1) After heating some wall materials (dispersions), the antioxidant properties can be enhanced, the oxidation of active ingredient can be inhibited, and product stability can be improved.
(2) Controlling the ratio of the first wall material to the second wall material within a specific range can enhance product stability while maintaining good water solubility, and can prepare microcapsules with good stability and excellent water solubility.

### Specific Models for Carrying Out the Present Invention

The embodiments of the present invention will be described in detail below with reference to examples, but those skilled in the art will understand that the following examples are only used to illustrate the present invention and should not be regarded as limiting the scope of the present invention. If the specific conditions are not specified in the examples, the conditions should be carried out according to the conventional conditions or the conditions recommended by the manufacturers. If the manufacturers of the reagents or instruments used are not indicated, they are all conventional products that can be purchased commercially.

The detection methods for the content of each active ingredient in the microcapsules of the following examples and comparative examples are shown in Table 1.

TABLE 1

| Name of active substance | Detection method |
|---|---|
| Oxidized Coenzyme Q10 | USP-NF2021 [symbol] Ubidecarenone [symbol] |
| Reduced Coenzyme Q10 | USP-NF2021 [symbol] Ubiquinol [symbol] |
| Vitamin D3 | EP10.0 [symbol] Cholecalciferol |
| | concentrate (powder form) [symbol] |
| Vitamin K2 | USP-NF2021 [symbol] Preparation [symbol] |
| Docosahexaenoic acid | GB 26400-2011, “Docosahexaenoic |
| glyceride | acid oil (fermentation |
| | method)” |
| Eicosatetraenonic acid | GB 26401-2011, “Food |
| glyceride | additive arachidonic acid oil |
| | (fermentation method)” |
| β-Carotene | GB 28310-2012, “Food additive |
| | β-carotene (fermentation |
| | method)” |
| canthaxanthin | NY_T 2896-2016 |
| NADH | Detected using high performance |
| | liquid chromatography, wherein, |
| | Mobile phase A: 0.1% trifluoroacetic acid, |
| | Mobile phase B: HPLC grade methanol; |
| | Chromatographic column: Agilent |
| | SB-C18 5um 250 × 4.6 mm; |
| | Detector: UV = 260 nm; |
| | Column temperature: 25° C.; |
| | Flow rate: 0.5 mL/min; |
| | Injection volume: 5 μL; |
| | Gradient elution conditions: |
| | time/min (0, 6.7, 13, 14.2, 15, 21), |
| | mobile phase A/vt % (100, 96, |
| | 92, 92, 100, 100), mobile phase |
| | B/vt % (0,4, 18, 18, 0, 0). |
| NMN | Detected using high performance |
| | liquid chromatography, wherein, |
| | Mobile phase A: 50 mmol/L |
| | potassium dihydrogen phosphate, |
| | Mobile phase B: acetonitrile; |
| | Chromatographic column: |
| | Agilent Zorbax SB-AQ, |
| | 250 × 4.6 mm; |
| | Detector: UV = 254 nm; |
| | Column temperature: 25° C.; |
| | Flow rate: 1.0 mL/min; |
| | Injection volume: 5 μL; |
| | Gradient elution conditions: time/min |
| | (0, 4, 5, 6, 10), mobile |
| | phase A/vt % (96, 80, 80, 96, 96), |
| | mobile phase B/vt % (4, 20, |
| | 20, 4, 4). |

### EXAMPLE 1: PREPARATION OF REDUCED COENZYME Q10 MICROCAPSULES

40 g of reduced coenzyme Q10 crystal (reduced coenzyme Q10 content was 99.5%) was taken and weighed, added with 1.5 g of glyceryl monostearate, 0.1 g of ascorbyl palmitate, 0.05 g of rosemary, stirred under vacuum and nitrogen supplementation at 60° C. to fully disperse and dissolve all components to obtain a core material.

17 g of sodium octenyl starch succinate, 5.5 g of sodium caseinate, 5.9 g of solid corn syrup, and 0.75 g of sodium ascorbate were taken and added into 30 ml of water, heated to 60° C. and stirred for 30 minutes, then heated to 110° C. and stirred for 10 minutes to form a first wall material-containing solution. It was cooled to room temperature.

22.5 g of sodium octenyl starch succinate, 5.95 g of solid corn syrup, and 0.75 g of sodium ascorbate were taken and added into 30 ml of water, stirred for dispersion and dissolution to form a second wall material-containing solution.

The first wall material-containing solution was added into a reaction kettle, the high-speed shearing machine was turned on with a rotation speed of 9000 r/min, the core material was slowly added, and high-speed shearing and emulsification were carried out for 10 minutes. Then the second wall material-containing solution was added and the high-speed shearing and emulsification were continued. After 10 minutes, the obtained emulsion was fed into a spray drying tower for granulation and drying. The inlet air temperature of the spray tower was 180° C. and the outlet air temperature was 80° C. 95.6 g of reduced coenzyme Q10 microcapsules were obtained. The content of reduced coenzyme Q10 in the microcapsules was detected to be 40.66%.

### EXAMPLE 2: PREPARATION OF OXIDIZED COENZYME Q10 MICROCAPSULES

45 g of oxidized coenzyme Q10 crystal (oxidized coenzyme Q10 content was 99.8%) was taken and weighed, added with 1.5 g of glyceryl monooleate and 0.5 g of ascorbyl palmitate, stirred under vacuum and stir with nitrogen supplementation at 60° C. to fully disperse and dissolve all components to obtain a core material.

18 g of sodium octenyl starch succinate, 6 g of sodium caseinate, 4 g of maltose syrup, and 0.5 g of sodium ascorbate were added to 30 ml of water, heated to elevate the temperature to 60° C. and stirred for 60 minutes, then heated to elevate the temperature to 100° C. and stirred for 15 minutes to form a first wall material-containing solution. It was cooled to room temperature.

24 g of sodium octenyl starch succinate and 0.5 g of sodium ascorbate were taken and added into 30 ml of water, stirred for dispersion and dissolution to form a second wall material-containing solution.

The first wall material-containing solution was added into a reaction kettle, the high-speed shearer was turned on with a rotation speed of 9000 r/min, the core material was slowly added, and high-speed shearing and emulsification were performed for 20 minutes. Then the second wall material-containing solution was added and the high-speed shearing and emulsification were continued. After 5 minutes, the obtained emulsion was fed into a spray drying tower for granulation and drying. The inlet air temperature of the spray tower was 180° C. and the outlet air temperature was 80° C. 96.3 g of oxidized coenzyme Q10 microcapsules were obtained. The content of oxidized coenzyme Q10 in the microcapsules was detected to be 45.26%.

### EXAMPLE 3: PREPARATION OF VITAMIN D3 MICROCAPSULES

12.5 g of vitamin D3 oil (vitamin D3 content was 4 million IU/g) was taken and weighed, added with 1.5 g of Span-60 and 0.5 g of α-tocopherol, stirred under vacuum and nitrogen supplementation at 50° C. to fully disperse and dissolve all components to obtain a core material.

7 g of sodium caseinate, 14 g of lactose, and 0.3 g of sodium ascorbate were added to 15 ml of water, heated to raise the temperature to 80° C. and stirred for 30 minutes, then heated to raise the temperature to 115° C. and stirred for 5 minutes to form a first wall material-containing solution. It was cooled to room temperature.

21 g of sodium caseinate, 42 g of lactose and 1.2 g of sodium ascorbate were taken and added into 45 ml of water, stirred for dispersion and dissolution to form a second wall material-containing solution.

The first wall material-containing solution was added into a reaction kettle, the high-speed shearing machine was turned on with a rotation speed of 10000 r/min, the core material was slowly added, and high-speed shearing and emulsification were performed for 45 minutes. Then the second wall material-containing solution was added and the high-speed shearing and emulsification were continued. After 10 minutes, the obtained emulsion was fed into a spray drying tower for granulation and drying. The inlet air temperature of the spray tower was 180° C. and the outlet air temperature was 80° C. 96.4 g of vitamin D3 microcapsules were obtained. The content of vitamin D3 in the microcapsules was detected to be 503,300 IU/g.

### EXAMPLE 4: PREPARATION OF VITAMIN K2 MICROCAPSULES

50 g of vitamin K2 oil (MK-7 content was 2%) was taken and weighed, added with 1 g of triglycerol monostearate and 0.5 g of rosemary extract, and stirred under vacuum and nitrogen supplementation at 55° C. to fully disperse and dissolve all components to obtain a core material.

26 g of sodium caseinate, 6 g of galactose, and 0.3 g of tea polyphenols were taken and added into 40 ml of water, heated to raise the temperature to 55° C. and stirred for 180 minutes, then heated to raise the temperature to 100° C. and stirred for 10 minutes to form a first wall material-containing solution. It was cooled to room temperature.

13 g of sodium caseinate, 3 g of galactose, and 0.2 g of tea polyphenols were taken and added into 20 ml of water, stirred for dispersion and dissolution to form a second wall material-containing solution.

The first wall material-containing solution was added into a reaction kettle, the high-speed shearing machine was turned on with a rotation speed of 12000 r/min, the core material was slowly added, and high-speed shearing and emulsification were performed for 15 minutes, then the second wall material-containing solution was added, and the high-speed shearing and emulsification were continued. After 30 minutes, the obtained emulsion was fed into a spray drying tower for granulation and drying. The inlet air temperature of the spray tower was 180° C. and the outlet air temperature was 80° C. 95.5 g of vitamin K2 microcapsules was obtained. The content of vitamin K2 in the microcapsules was detected to be 10.38%.

### EXAMPLE 5: PREPARATION OF β-CAROTENE MICROCAPSULES

10 g of β-carotene crystal (β-carotene content was 99.5%) was taken and weighed, added with 10 g of soybean oil, 0.5 g of decaglyceryl decaoleate, and 0.5 g of rosemary extract, and stirred under vacuum and nitrogen supplementation at 175° C. to fully disperse and dissolve all components to obtain a core material.

10 g of whey protein, 15 g of xylose, and 1.5 g of tea polyphenols were taken and added into 20 ml of water, heated to raise the temperature to 65° C. and stirred for 120 minutes, then heated to raise the temperature to 120° C. and stirred for 5 minutes to form a first wall material-containing solution. It was cooled to room temperature.

20 g of whey protein, 30 g of xylose and 2.5 g of tea polyphenols were taken and added into 40 ml of water, stirred for dispersion and dissolution to form a second wall material-containing solution.

The first wall material-containing solution was added into a reaction kettle, the high-speed shearing machine was turned on with a rotation speed of 12000 r/min, the core material was slowly added, and high-speed shearing and emulsification were performed for 10 minutes, then the second wall material-containing solution was added, and the high-speed shearing and emulsification were continued. After 5 minutes, the obtained emulsion was fed into a spray drying tower for granulation and drying. The inlet air temperature of the spray tower was 180° C. and the outlet air temperature was 70° C. 97.2 g of β-carotene microcapsules were obtained. The content of β-carotene in the microcapsules was detected to be 9.96%.

### EXAMPLE 6: PREPARATION OF CANTHAXANTHIN MICROCAPSULES

10.5 g of canthaxanthin crystal (canthaxanthin content was 95%) was taken and weighed, added with 19.5 g of sunflower oil and 2 g of soybean lecithin, and stirred under vacuum and nitrogen supplementation at 210° C. to fully disperse and dissolve all components to obtain a core material.

18 g of whey protein, 15 g of mannose, and 1 g of tea polyphenols were taken and added into 30 ml of water, heat to raise the temperature to 60° C. and stirred for 150 minutes, then heated to raise the temperature to 115° C. and stirred for 10 minutes to form a first wall material-containing solution. It was cooled to room temperature.

18 g of sodium octenyl starch succinate, 15 g of mannose, and 1 g of tea polyphenols were taken and added into 40 ml of water, and stirred for dispersion and dissolution to form a second wall material-containing solution.

The first wall material-containing solution was added into a reaction kettle, the high-speed shearing machine was turned on with a rotation speed of at 9000 r/min, the core material was slowly added, and high-speed shearing and emulsification were performed for 30 minutes. Then the second wall material-containing solution was added, and the high-speed shearing and emulsification were continued. After 10 minutes, the obtained emulsion was fed into a spray drying tower for granulation and drying. The inlet air temperature of the spray tower was 180° C. and the outlet air temperature was 70° C. 95.6 g of canthaxanthin microcapsules were obtained. The content of canthaxanthin in the microcapsules was detected to be 10.16%.

### EXAMPLE 7: PREPARATION OF DOCOSAHEXAENOIC ACID MICROCAPSULES

25 kg of docosahexaenoic acid algal oil (docosahexaenoic acid content was 40%) was taken and weighed, added with 2 kg of lecithin, and stirred under vacuum and nitrogen supplementation at 40° C. to fully disperse and dissolve all components to obtain a core material.

9 kg of soy protein, 34 kg of fructose, and 3 kg of tea polyphenols were taken and added into 40 L of water, heated to raise the temperature to 50° C. and stirred for 60 minutes, then heated to raise the temperature to 110° C. and stirred for 15 minutes to form a first wall material-containing solution. It was cooled to room temperature.

27 kg of sodium octenyl starch succinate was taken and added into 20 L of 40° C. water, and stirred for dispersion and dissolution to form a second wall material-containing solution.

The first wall material-containing solution was added into a reaction kettle, the high-speed shearing machine was turn on with a rotation speed of 9000 r/min, the core material was slowly added, and high-speed shearing and emulsification were performed for 40 minutes. Then the second wall material-containing solution was added, and the high-speed shearing and emulsification were continued. After 10 minutes, high pressure homogenization was performed three times at 25 MPa. The obtained emulsion was fed into a spray drying tower for granulation and drying. The inlet air temperature of the spray tower was 180° C. and the outlet air temperature was 85° C. 96.2 kg of docosahexaenoic acid microcapsules was obtained. The content of docosahexaenoic acid in the microcapsules was detected to be 10.68%.

### EXAMPLE 8: PREPARATION OF EICOSATETRAENOIC ACID MICROCAPSULES

25 kg of eicosatetraenoic acid oil (eicosatetraenoic acid content was 40%) was taken and added with 1.5 kg of glyceryl monostearate, and stirred under vacuum and nitrogen supplementation at 40° C. to fully disperse and dissolve all components to obtain a core material.

10 kg of corn protein, 30 kg of isomaltulose, and 2 kg of sodium erythorbate were taken and added into 40 L of water, heated to raise the temperature to 55° C. and stirred for 90 minutes, then heated to raise the temperature to 120° C. and stirred for 5 minutes to form a first wall material-containing solution. It was cooled to room temperature.

20 kg of sodium octenyl starch succinate was taken and added into 20 L of water, and stirred for dispersion and dissolution to form a second wall material-containing solution.

The first wall material-containing solution was added into a reaction kettle, the high-speed shearing machine was turned on with a rotation speed of 9000 r/min, the core material was slowly added, and high-speed shearing and emulsification were performed for 35 minutes. Then the second wall material-containing solution was added, and the high-speed shearing and emulsification were continued. After 15 minutes, the obtained emulsion was fed into a spray fluidized bed drying tower for granulation and drying, in which the fluidized bed is distributed with fluidized corn starch and tricalcium phosphate (the mass ratio of the two was 1:0.15), to obtain 98.6 kg of double-coated eicosatetraenoic acid microcapsules. The content of eicosatetraenoic acid in the microcapsules was detected to be 10.21%.

### EXAMPLE 9: PREPARATION OF NADH MICROCAPSULES

10 g of reduced β-nicotinamide adenine dinucleic acid disodium salt (NADH content was 99%) was taken and weighed, added with 10 g of water, and stirred for dissolution to obtain a NADH solution; 30 g of corn oil was added with 1.5 g of glyceryl monostearate and 0.15 g of ascorbyl palmitate, fully dissolved and stirred evenly, then added with the NADH solution, and stirred under vacuum and nitrogen supplementation at 55° C. and high speed of 10000 r/min for 10 minutes to obtain a core material.

17 g of sodium octenyl starch succinate, 5.5 g of sodium caseinate, 5.9 g of solid corn syrup, and 0.75 g of sodium ascorbate were taken and added into 30 ml of water, heated to 60° C. and stirred for 30 minutes, then heated to 110° C. and stirred for 10 minutes to form a first wall material-containing solution. It was cooled to room temperature.

22.5 g of sodium octenyl starch succinate, 5.95 g of solid corn syrup, and 0.75 g of sodium ascorbate were taken and added into 30 ml of water, and stirred for dispersion and dissolution to form a second wall material-containing solution.

The first wall material-containing solution was added into a reaction kettle, the high-speed shearing machine was turned on with a rotation speed of 9000 r/min, the core material was slowly added, and high-speed shearing and emulsification were performed for 10 minutes. Then the second wall material-containing solution was added, and the high-speed shearing and emulsification were continued. After 10 minutes, the obtained emulsion was fed into a spray drying tower for granulation and drying. The inlet air temperature of the spray tower was 160° C. and the outlet air temperature was 80° C. 96.7 g of NADH microcapsules was obtained. The NADH content in the microcapsules was detected to be 10.16%.

### EXAMPLE 10: PREPARATION OF NMN MICROCAPSULES

15 g of NMN crystal (NMN content was 98%) was taken and weighed, added with 15 g of water, and stirred for dissolution to obtain a NMN solution; 1.5 g of Span-80 and 0.5 g of γ-tocopherol were added to 35 g of MCT, fully dissolved and stirred evenly, and then added with the NMN solution, and stirred under vacuum and nitrogen supplementation at 60° C. and high speed of 10000 r/min for 10 minutes to obtain a core material.

18 g of sodium octenyl starch succinate, 6 g of sodium caseinate, 4 g of maltose syrup, and 0.5 g of sodium ascorbate were taken, added into 30 ml of water, heated to raise the temperature to 60° C. and stirred for 60 min, then heated to raise the temperature to 100° C. and stirred for 15 min to form a first wall material-containing solution. It was cooled to room temperature.

24 g of sodium octenyl starch succinate and 0.5 g of sodium ascorbate were taken and added into 30 ml of water, and stirred for dispersion and dissolution to form a second wall material-containing solution.

The first wall material-containing solution was added into a reaction kettle, the high-speed shearing machine was turned on with a rotation speed of 9000 r/min, the core material was slowly added, and high-speed shearing and emulsification were performed for 20 minutes. Then the second wall material-containing solution was added, and the high-speed shearing and emulsification were continued. After 5 minutes, the obtained emulsion was fed into a spray drying tower for granulation and drying. The inlet air temperature of the spray tower was 160° C. and the outlet air temperature was 80° C. 98.6 g of NMN microcapsules was obtained. The NMN content in the microcapsules was detected to be 15.39%.

### COMPARATIVE EXAMPLE 1

40 g of reduced coenzyme Q10 crystal (reduced coenzyme Q10 content was 99.5%) was taken and weighed, added with 1.5 g of glyceryl monostearate, 0.1 g of ascorbyl palmitate, and 0.05 g of rosemary, and stirred under vacuum and nitrogen supplementation at 60° C. to fully disperse and dissolve all components to obtain a core material.

17 g of sodium octenyl starch succinate, 5.5 g of sodium caseinate, 5.9 g of solid corn syrup, and 0.75 g of sodium ascorbate were taken and added into 30 ml of water, heated to raise the temperature to 60° C. and stirred for 40 minutes to form a first wall material-containing solution. It was cooled to room temperature.

22.5 g of sodium octenyl starch succinate, 5.95 g of solid corn syrup, and 0.75 g of sodium ascorbate were taken and added into 30 ml of water, and stirred for dispersion and dissolution to form a second wall material-containing solution.

The first wall material-containing solution was added into a reaction kettle, the high-speed shearing machine was turned on with a rotation speed of 9000 r/min, the core material was slowly added, and high-speed shearing and emulsification were performed for 10 minutes. Then the second wall material-containing solution was added, and the high-speed shearing and emulsification were continued. After 10 minutes, the obtained emulsion was fed into a spray drying tower for granulation and drying. The inlet air temperature of the spray tower was 180° C. and the outlet air temperature was 80° C. 95.9 g of reduced coenzyme Q10 microcapsules was obtained. The content of reduced coenzyme Q10 in the microcapsules was detected to be 39.57%.

### COMPARATIVE EXAMPLE 2

40 g of reduced coenzyme Q10 crystal (reduced coenzyme Q10 content was 99.5%) was taken and weighed, added with 1.5 g of glyceryl monostearate, 0.1 g of ascorbyl palmitate, and 0.05 g of rosemary, and stirred under vacuum and nitrogen supplementation at 60° C. to fully disperse and dissolve all components to obtain a core material.

17 g of sodium octenyl starch succinate, 5.5 g of sodium caseinate, 5.9 g of solid corn syrup, and 0.75 g of sodium ascorbate were taken and added into 30 ml of water, heated to raise the temperature to 110° C. and stirred for 40 minutes to form a first wall material-containing solution. It was cooled to room temperature.

22.5 g of sodium octenyl starch succinate, 5.95 g of solid corn syrup, and 0.75 g of sodium ascorbate were taken and added into 30 ml of water, and stirred for dispersion and dissolution to form a second wall material-containing solution.

The first wall material-containing solution was added into a reaction kettle, the high-speed shearing machine was turned on with a rotation speed of 9000 r/min, the core material was slowly added, and high-speed shearing and emulsification were performed for 10 minutes. Then the second wall material-containing solution was added, and the high-speed shearing and emulsification were continued. After 10 minutes, the obtained emulsion was fed into a spray drying tower for granulation and drying. The inlet air temperature of the spray tower was 180° C. and the outlet air temperature was 80° C. 96.3 g of reduced coenzyme Q10 microcapsules was obtained. The content of reduced coenzyme Q10 in the microcapsules was detected to be 39.63%.

### COMPARATIVE EXAMPLE 3

40 g of reduced coenzyme Q10 crystal (reduced coenzyme Q10 content was 99.5%) was taken and weighed, added with 1.5 g of glyceryl monostearate, 0.1 g of ascorbyl palmitate, 0.05 g of rosemary, and stirred under vacuum and nitrogen supplementation at 60° C. to fully disperse and dissolve all components to obtain a core material.

17 g of sodium octenyl starch succinate, 5.5 g of sodium caseinate, 5.9 g of solid corn syrup, and 0.75 g of sodium ascorbate were taken and added into 30 ml of water, heated to raise the temperature to 110° C. and stirred for 10 min, then cooled to lower the temperature to 60° C. and stirred for 30 min to form a first wall material-containing solution. It was cooled to room temperature.

22.5 g of sodium octenyl starch succinate, 5.95 g of solid corn syrup, and 0.75 g of sodium ascorbate were taken and added into 30 ml of water, and stirred for dispersion and dissolution to form a second wall material-containing solution.

The first wall material-containing solution was added into a reaction kettle, the high-speed shearing machine was turned on with a rotation speed of 9000 r/min, the core material was slowly added, and high-speed shearing and emulsification were performed for 10 minutes. Then the second wall material-containing solution was added, and the high-speed shearing and emulsification were continued. After 10 minutes, the obtained emulsion was fed into a spray drying tower for granulation and drying. The inlet air temperature of the spray tower was 180° C. and the outlet air temperature was 80° C. 95.5 g of reduced coenzyme Q10 microcapsules was obtained. The content of reduced coenzyme Q10 in the microcapsules was detected to be 39.26%.

### COMPARATIVE EXAMPLE 4

40 g of reduced coenzyme Q10 crystal (reduced coenzyme Q10 content was 99.5%) was taken and weighed, added with 1.5 g of glyceryl monostearate, 0.1 g of ascorbyl palmitate, and 0.05 g of rosemary, and stirred under vacuum and nitrogen supplementation at 60° C. to fully disperse and dissolve all components to obtain a core material.

17 g of sodium octenyl starch succinate, 5.5 g of sodium caseinate, 5.9 g of corn syrup, and 0.75 g of sodium ascorbate were taken and added 30 ml of water, and stirred for dispersion and dissolution to form a first wall material-containing solution.

22.5 g of sodium octenyl starch succinate, 5.95 g of solid corn syrup, and 0.75 g of sodium ascorbate were taken and added into 30 ml of water, and stirred for dispersion and dissolution to form a second wall material-containing solution.

The first wall material-containing solution was added into a reaction kettle, the high-speed shearing machine was turned on with a rotation speed of 9000 r/min, the core material was slowly added, and high-speed shearing and emulsification were performed for 10 minutes, then the second wall material-containing solution was added, and the high-speed shearing and emulsification were continued. After 10 minutes, the obtained emulsion was fed into a spray drying tower for granulation and drying. The inlet air temperature of the spray tower was 180° C. and the outlet air temperature was 80° C. 95.3 g of reduced coenzyme Q10 microcapsules was obtained. The content of reduced coenzyme Q10 in the microcapsules was detected to be 38.03%.

### COMPARATIVE EXAMPLE 5

40 g of reduced coenzyme Q10 crystal (reduced coenzyme Q10 content was 99.5%) was taken and weighed, added with 1.5 g of glyceryl monostearate, 0.1 g of ascorbyl palmitate, and 0.05 g of rosemary, and stirred under vacuum and nitrogen supplementation at 60° C. to fully disperse and dissolve all components to obtain a core material.

39.5 g of sodium octenyl starch succinate, 5.5 g of sodium caseinate, 11.85 g of solid corn syrup, and 1.5 g of sodium ascorbate were taken and added into 60 ml of water, heated to raise the temperature to 60° C. and stirred for 30 minutes, then heated to raise the temperature to 110° C. and stirred for 10 minutes to form a wall material-containing solution. It was cooled to room temperature.

The wall material-containing solution was added into a reaction kettle, the high-speed shearing machine was turned on with a rotation speed of 9000 r/min, the core material was slowly added, and high-speed shearing and emulsification were performed for 20 minutes. The obtained emulsion was fed into a spray drying tower for granulation and drying. The inlet air temperature of the spray drying tower was 180° C., and the outlet air temperature was 80° C. 95.4 g of reduced coenzyme Q10 microcapsules was obtained, and the content of reduced coenzyme Q10 in the microcapsules was detected to be 38.92%.

### COMPARATIVE EXAMPLE 6

40 g of reduced coenzyme Q10 crystal (reduced coenzyme Q10 content was 99.5%) was taken and weighed, added with 1.5 g of glyceryl monostearate, 0.1 g of ascorbyl palmitate, and 0.05 g of rosemary, and stirred under vacuum and nitrogen supplementation at 60° C. to fully disperse and dissolve all components to obtain a core material.

39.5 g of sodium octenyl starch succinate, 5.5 g of sodium caseinate, 11.85 g of solid corn syrup, and 1.5 g of sodium ascorbate were taken and added into 60 ml of water, heated to raise the temperature to 50° C. and stirred for 40 minutes to form a wall material-containing solution. It was cooled to room temperature.

The wall material-containing solution was added into a reaction kettle, the high-speed shearing machine was turned on with a rotation speed of 9000 r/min, the core material was slowly added, and high-speed shearing and emulsification were performed for 20 minutes. The obtained emulsion was fed into a spray drying tower for granulation and drying. The inlet air temperature of the spray drying tower was 180° C., and the outlet air temperature was 80° C. 95.2 g of reduced coenzyme Q10 microcapsules was obtained, and the content of reduced coenzyme Q10 in the microcapsules was detected to be 39.24%.

### Example for Detection of Stability

For each of the microcapsules prepared in the above Examples and Comparative Examples, 4 samples were taken and vacuum sealed in aluminum foil bags, respectively, and placed in a 50° C. incubator for accelerated aging experiment. One sample was used as a control, one sample was placed for 2 weeks, one sample was placed for 4 weeks, and one sample was placed for 8 weeks. The content decline was detected to examine the stability of microcapsules during the accelerated aging experiment. The results were shown in Table 2.

TABLE 2

| | | Decline | Decline | Decline |
|---|---|---|---|---|
| | Initial | within 2 | within 4 | within 8 |
| Sample | content | weeks/% | weeks/% | weeks/% |
| Reduced coenzyme | 40.66% | 0.25 | 0.66 | 1.14 |
| Q10 microcapsules | | | | |
| of Example 1 | | | | |
| Oxidized coenzyme | 45.26% | 0.16 | 0.43 | 0.89 |
| Q10 microcapsules | | | | |
| of Example 2 | | | | |
| Vitamin D3 | 503300 | 0.71 | 1.44 | 3.34 |
| microcapsules of Example 3 | IU/g | | | |
| Vitamin K2 | 10.38% | 0.01 | 0.05 | 0.08 |
| microcapsules of Example 4 | | | | |
| β-Carotene microcapsules | 9.96% | 0.54 | 1.86 | 3.62 |
| of Example 5 | | | | |
| Canthaxanthin | 10.16% | 0.46 | 1.26 | 2.78 |
| microcapsules of | | | | |
| Example 6 | | | | |
| Docosahexaenoic | 10.68% | 0.02 | 0.02 | 0.06 |
| acid microcapsules of | | | | |
| Example 7 | | | | |
| Eicosatetraenoic | 10.21% | 0.01 | 0.02 | 0.06 |
| acid microcapsules of | | | | |
| Example 8 | | | | |
| NADH microcapsules | 10.16% | 0.27 | 0.64 | 1.25 |
| of Example 9 | | | | |
| NMN microcapsules | 15.39% | 0.13 | 0.42 | 0.91 |
| of Example 10 | | | | |
| Reduced coenzyme | 39.57% | 0.86 | 2.87 | 5.49 |
| Q10 microcapsules | | | | |
| of Comparative Example 1 | | | | |
| Reduced coenzyme | 39.63% | 0.79 | 2.45 | 5.22 |
| Q10 microcapsules | | | | |
| of Comparative Example 2 | | | | |
| Reduced coenzyme | 39.26% | 0.80 | 2.63 | 5.32 |
| Q10 microcapsules | | | | |
| of Comparative Example 3 | | | | |
| Reduced coenzyme | 38.03% | 1.23 | 4.04 | 7.53 |
| Q10 microcapsules | | | | |
| of Comparative Example 4 | | | | |
| Reduced coenzyme | 38.92% | 1.06 | 3.38 | 6.97 |
| Q10 microcapsules | | | | |
| of Comparative Example 5 | | | | |
| Reduced coenzyme | 39.24% | 0.82 | 2.68 | 5.40 |
| Q10 microcapsules | | | | |
| of Comparative Example 6 | | | | |

### Example for Detection Water Solubility

100 mL of tap water was taken and kept at the temperature of 15° C., added with 1 gram of the microcapsules prepared in one of the above Examples and Comparative Examples. The time for the microcapsules to completely disperse and dissolve in the standing water to form a uniform and stable emulsion was observed. The results were shown in the Table 3.

TABLE 3

| Sample | Dissolution time |
|---|---|
| Reduced coenzyme Q10 microcapsules of Example 1 | 5 min 10 s |
| Oxidized coenzyme Q10 microcapsules of Example 2 | 6 min 23 s |
| Vitamin D3 microcapsules of Example 3 | 1 min 30 s |
| Vitamin K2 microcapsules of Example 4 | 6 min 15 s |
| β-Carotene microcapsules of Example 5 | 2 min 38 s |
| Canthaxanthin microcapsules of Example 6 | 4 min 10 s |
| Docosahexaenoic acid microcapsules of Example 7 | 3 min 22 s |
| Eicosatetraenoic acid microcapsules of Example 8 | 3 min 24 s |
| NADH microcapsules of Example 9 | 4 min 55 s |
| NMN microcapsules of Example 10 | 4 min 56 s |
| Reduced coenzyme Q10 microcapsules | 5 min 19 s |
| of Comparative Example 1 | |
| Reduced coenzyme Q10 microcapsules | 5 min 53 s |
| of Comparative Example 2 | |
| Reduced coenzyme Q10 microcapsules | 5 min 27 s |
| of Comparative Example 3 | |
| Reduced coenzyme Q10 microcapsules | 4 min 46 s |
| of Comparative Example 4 | |
| Reduced coenzyme Q10 microcapsules | 8 min 21 s |
| of Comparative Example 5 | |
| Reduced coenzyme Q10 microcapsules | 5 min 49 s |
| of Comparative Example 6 | |

Although specific embodiments of the present invention have been described in detail, those skilled in the art would understand, according to all the teachings that have been disclosed, various modifications and substitutions can be made to those details, and these changes are within the protection scope of the present invention. The full scope of the present invention is given by the appended claims and any equivalents thereof.

What is claimed is:

1. A method for preparing a microcapsule, in which
the microcapsule comprises a wall material and a core material, and the wall material comprises a first wall material and a second wall material;
the method comprises a step of preparing a first dispersion containing the first wall material and a second dispersion containing the second wall material;
wherein,
the first dispersion is subjected to a heat treatment, the heat treatment comprises a first heating stage and a second heating stage, and the temperature of the first heating stage is lower than the temperature of the second heating stage; and
the second dispersion is not subjected to a heat treatment, or is subjected to a heat treatment at a temperature of not more than 50° C., or the second dispersion is subjected to a heat treatment at a temperature that is lower than the temperature of the heat treatment to which the first dispersion is subjected.

2. The preparation method according to claim 1, wherein the weight of the first wall material is less than or equal to the weight of the second wall material.

3. The preparation method according to claim 1, wherein the weight ratio of the first wall material to the second wall material is 1:(0.5-3), preferably 1:(1-3), 1:(1.2-3) or 1:(1.5-3).

4. The preparation method according to claim 1, wherein the heat treatment to which the first dispersion is subjected is performed at a temperature of 50° C. to 120° C., 50° C. to 90° C., 50° C. to 80° C., or 55° C. to 65° C.

5. The preparation method according to claim 1, wherein,
the heating time of the first heating stage is longer than the heating time of the second heating stage.

6. The preparation method according to claim 1, wherein:
the temperature of the first heating stage is 50° C. to 90° C., 50° C. to 80° C., or 55° C. to 65° C.; and/or
the temperature of the second heating stage is 90° C. to 120° C., 90° C. to 110° C., 100° C. to 120° C., 105° C. to 115° C., 110° C. to 120° C., or 100° C. to 110° C.

7. The preparation method according to claim 1, wherein,
the heating time of the first heating stage is 30 to 180 minutes, 30 to 120 minutes, 30 to 90 minutes, 30 to 60 minutes, 60 to 90 minutes, or 25 to 35 minutes; and/or
the heating time of the second heating stage is 5 to 25 minutes, 5 to 20 minutes, 5 to 15 minutes, 5 to 10 minutes, 10 to 25 minutes, 10 to 20 minutes, 10 to 15 minutes, or 8 to 12 minutes.

8. The preparation method according to claim 1, wherein,
the temperature of the first heating stage is 55° C. to 65° C., and the temperature of the second heating stage is 105° C. to 115° C.; and
the heating time of the first heating stage is 25 to 35 minutes, and the heating time of the second heating stage is 8 to 12 minutes.

9. The preparation method according to claim 1, wherein the wall material is composed of a first wall material and a second wall material;
preferably, the first wall material and the second wall material are the same or different;
preferably, the wall material of the microcapsule accounts for 40% to 99%, preferably 50% to 85%, and more preferably 60% to 70% of the weight of the microcapsule.

10. The preparation method according to claim 1, wherein the first wall material and the second wall material independently comprise a protein-based wall material and/or a carbohydrate-based wall material;
preferably, the protein-based wall material is one or more selected from the group consisting of alkali metal caseinate, whey protein, soy protein, wheat protein and corn protein; preferably, the alkali metal caseinate is one or more selected from the group consisting of sodium caseinate, potassium caseinate and calcium caseinate;
preferably, the carbohydrate-based wall material is one or more selected from the group consisting of starch, glucose, xylose, maltodextrin, maltose syrup, fructose, galactose, lactose, corn syrup, mannose and isomaltulose; preferably, the starch is a corn starch; preferably, the starch is a modified starch; preferably, the modified starch is one or more selected from the group consisting of sodium octenyl starch succinate and calcium octenyl starch succinate;
preferably, the first wall material and the second wall material further independently comprise a first antioxidant; preferably, the first antioxidant is a water-soluble antioxidant, preferably is one or more selected from the group consisting of tea polyphenols, ascorbic acid, erythorbic acid, sodium ascorbate and sodium erythorbate.

11. The preparation method according to claim 1, wherein the first dispersion and the second dispersion are independently solutions or emulsions.

12. The preparation method according to claim 1, wherein the core material comprises an active ingredient, such as a water-soluble active ingredient and/or a fat-soluble active ingredient;
preferably, the active ingredient is a nutrient, such as a water-soluble nutrient and/or a fat-soluble nutrient;
preferably, the core material further comprises one or more selected from the group consisting of emulsifier, second antioxidant and carrier oil.

13. The preparation method according to claim 12, wherein,
the fat-soluble nutrient is one or more selected from the group consisting of fat-soluble vitamin, fat-soluble quasi-vitamin, carotenoid, polyunsaturated fatty acid and monounsaturated fatty acid;
preferably, the fat-soluble vitamin is one or more selected from the group consisting of vitamin A, vitamin D, vitamin E and vitamin K;
preferably, the fat-soluble quasi-vitamin is at least one selected from the group consisting of coenzyme Q10, taurine, and carnitine; the coenzyme Q10 is one or more selected from the group consisting of oxidized coenzyme Q10 and reduced coenzyme Q10;
preferably, the carotenoid is one or more selected from the group consisting of β-carotene, β-zeaxanthin, astaxanthin, canthaxanthin, lycopene, lutein, nobiletin and zeaxanthin;
preferably, the polyunsaturated fatty acid is one or more selected from the group consisting of docosahexaenoic acid, docosapentaenoic acid, eicosapentaenoic acid, eicosatetraenoic acid, linoleic acid and linolenic acid;
preferably, the monounsaturated fatty acid is one or more selected from the group consisting of oleic acid, myristic acid, palmitoleic acid, trans-oleic acid, ricinoleic acid, erucic acid and cetoleic acid.

14. The preparation method according to claim 12, wherein,
the water-soluble nutrient is one or more selected from the group consisting of water-soluble vitamin, mineral ion, nattokinase, S-adenosylmethionine, glyceric acid, pyrroloquinoline quinone sodium salt, phosphatidylserine, glutathione, nicotinamide, nicotinamide adenine mononucleotide, nicotinamide ribose, and nicotinamide adenine dinucleotide;
preferably, the water-soluble vitamin is one or more selected from the group consisting of vitamin C and vitamin B family;
preferably, the vitamin B family is one or more selected from the group consisting of vitamin B1, vitamin B2, vitamin B3, vitamin B5, vitamin B6, folic acid, vitamin B12, niacin and lipoic acid; preferably, the folic acid is an active folic acid, such as calcium L-5-methyltetrahydrofolate;
preferably, the mineral ion is one or more selected from the group consisting of sodium ion, potassium ion, calcium ion, zinc ion, magnesium ion and iron ion;
preferably, the glutathione is one or more selected from the group consisting of oxidized glutathione and reduced glutathione;
preferably, the nicotinamide adenine mononucleotide is one or more selected from the group consisting of reduced β-nicotinamide mononucleotide and oxidized β-nicotinamide mononucleotide;
preferably, the nicotinamide adenine dinucleotide is one or more selected from the group consisting of reduced nicotinamide adenine dinucleotide and oxidized nicotinamide adenine dinucleotide;
preferably, the emulsifier is at least one selected from the group consisting of monoglycerides, Spans, polyglycerol esters and phospholipids; preferably, the monoglycerides are at least one selected from the group consisting of glyceryl monostearate, glyceryl monooleate, and glyceryl monolaurate;
the Spans are preferably at least one selected from the group consisting of Span-20, Span-40, Span-60, Span-80 and Span-85; the polyglycerol esters are preferably at least one selected from the group consisting of triglycerol monostearate, hexaglycerol monooleate and decaglycerol decaoleate; the phospholipids are preferably at least one selected from the group consisting of soy phospholipid, lecithin, cephalin and phosphatidylserine;
preferably, the second antioxidant is an oil-soluble antioxidant, preferably is one or more selected from the group consisting of tocopherol, ascorbyl palmitate, rosemary extract, phospholipid, butylhydroxyanisole, dibutylhydroxytoluene and tert-butylhydroquinone; the tocopherol is at least one selected from the group consisting of α-tocopherol, β-tocopherol, γ-tocopherol, δ-tocopherol and tocotrienol; and the phospholipid is at least one selected from the group consisting of soybean phospholipid, lecithin, cephalin and phosphatidylserine;
preferably, the carrier oil is selected from the group consisting of soybean oil, palm kernel oil, cottonseed oil, rapeseed oil, sunflower oil, coconut oil, corn oil, sesame oil, rice bran oil, castor oil, olive oil, flax oil, and safflower oil, peanut oil, and medium-chain fatty glyceride.

15. The preparation method according to any claim 1, which further comprises a step of preparing the microcapsule from the first dispersion, the second dispersion and the core material;
preferably, which comprises the following steps:
A. the first dispersion that has been subjected to the heat treatment is mixed with the core material, and undergoes emulsification;
B. the second dispersion that has been subjected or not been subjected to the heat treatment is added, and undergoes emulsification; and
C. granulation and drying are performed to obtain the microcapsule;
preferably, which further comprises the following step:
D. the microcapsule of step C is embedded to obtain a double-coated microcapsule.

16. The preparation method according to claim 15, wherein,
the emulsification in step A and step B is independently performed by high-speed shearing method, high-pressure homogenization method, micro-jet method or ultrasonic method; and/or
the granulation and drying in step C are performed by spray cooling method, spray drying method or spray fluidized bed drying method to obtain the microcapsule.

17. The preparation method according to claim 1, comprising the following steps:
(1) a first dispersion containing the first wall material and a second dispersion containing the second wall material are prepared;
wherein, the first dispersion is heated at 55° C. to 60° C. for 25 to 35 minutes, and then heated at 105° C. to 115° C. for 8 to 12 minutes; and the second dispersion is not subjected to heat treatment;
(2) the first dispersion is mixed with the core material, and undergoes emulsification;
(3) the second dispersion is added, and undergoes emulsification; and
(4) granulation and drying are performed to obtain the microcapsule;
wherein,
the core material comprises 35 to 45 parts by weight of reduced coenzyme Q10 and appropriate amounts of an emulsifier and an oil-soluble antioxidant;
the first dispersion comprises 15 to 20 parts by weight of sodium octenyl starch succinate, 4 to 7 parts by weight of sodium caseinate, and appropriate amounts of a carbohydrate-based wall material, an antioxidant and water; and
the second dispersion comprises 20 to 25 parts by weight of sodium octenyl starch succinate and appropriate amounts of a carbohydrate-based wall material, an antioxidant and water.

18. The preparation method according to claim 1, which further comprises the following step:
the prepared microcapsule is embedded twice to obtain a double-coated microcapsule.

19. A microcapsule, which is prepared by the preparation method according to claim 1.

20. A composition, which comprises the microcapsule according to claim 19 and one or more pharmaceutically or bromatologically acceptable auxiliary materials;
preferably, the composition is a tablet, soft capsule, hard capsule, powder or pill.
